(19) United States
(12) Patent Application Publication
(10) Pub. No.: US 2003/0229373 A1
(43) Pub. Date: Dec. 11, 2003

(54) Apparatus for preventing regeneration of endometrial synechia

(76) Inventor: Woo-Dong Lee, Daegu-City (KR)

Correspondence Address: Paul J. Farrell, Esq., DILWORTH & BARRESE, LLP, 333 Earle Ovington Blvd., Uniondale, NY, 11553, US

(21) Appl. No.: 10/457,989

(22) Filed: Jun. 10, 2003

(30) Foreign Application Priority Data
Jun. 11, 2002 (KR) 2002/32475

(51) Int. Cl. A61M 29/00

(52) U.S. Cl. 606/193

(57) ABSTRACT

An apparatus for preventing regeneration of endometrial synechia, which comprises: an air injection tube, into which air is injected from the outside; a drainage lumen tube for draining intrauterine residue and blood, in which the air injection tube is received; an air hole formed at one side around an upper end of the drainage lumen tube to eject the air passed through the air injection tube; and a balloon fixed to the drainage lumen tube while covering the air hole formed at one side around the upper end of the drainage lumen tube, the balloon being configured to be inflated to conform to a shape of a woman's uterus within the uterus by the air injected through the air injection tube and air hole. The balloon of the apparatus is adapted to uniformly and safely spread to the whole interior of the uterus including the intrauterine isthmus and fundus uteri, thereby preventing regeneration of endometrial synechia as well as minimizing inconvenience to patients. Furthermore, the apparatus allows a concurrent hormone treatment to be carried out to promote formation of new endometrium.

### PRIORITY

This application claims priority to an application entitled “Apparatus for Preventing Regeneration of Endometrial Synechia”, filed in the Korean Industrial Property Office on Jun. 11, 2002 and assigned Serial No. 2002-32475, the contents of which are hereby incorporated by reference.

### BACKGROUND OF THE INVENTION

1. Field of the Invention

The present invention relates to an apparatus for preventing regeneration of endometrial synechia, and more particularly to an apparatus for preventing regeneration of endometrial synechia, which comprises a drainage lumen tube for drainage of residue and a balloon configured to be inflated to conform to the shape of a woman's uterus by pressurized air passed through an air injection tube and air hole in order to maintain the existing shape of the uterus after ablating a uterine adhesion formed due to the disruption of endometrium, the apparatus allowing a concurrent hormone treatment using oestrogen(estradiol) acting to be carried out to promote formation of endometrium, thereby effectively preventing the uterine adhesion from being reformed at the endometrium.

2. Description of the Related Art

Generally, endometrium serves to protect a fertilized ovum growing in a matrix, and a woman's uterus has a slightly flat branched shape. The upper part of the uterus, having a sack shape, is known as the “corpus uteri”, and the lower part thereof, having a slender pinched shape, is known as the “cervix”. In addition, the lumen of the cervix is known as the “cervix uteri”.

The uterus is positioned between the urinary bladder and rectum within the pelvis, and leans slightly forward between the cervix and the corpus uteri. The corpus uteri extends upward from the ovaries to the abdominal cavity through a pair of left and right oviducts positioned at the upper side thereof, and also extends downward into the vagina through the cervix.

The inner surface of the uterus is lined with a mucous membrane, a so-called uterine mucous membrane or endometrium. This endometrium has a thickness of 1.5 to 2 millimeters, and contains glandular cells, such as uterine gland cells and cervical gland cells, under epithelial cells which makeup an uppermost superficial layer. The glandular cells provide a secretion required for self-cleansing and protection of the uterine lumen, and this secretion also is one component of physiological elytrorrhea. In the case of the mature uterus, the cells of the endometrium periodically undergo cycles of cell proliferation and cell death under the stimulation of anterior pituitary hormones and ovarian hormones. This is a physiological response to the implantation of a fertilized ovum. If there is no implantation of a fertilized ovum, the endometrium substitutes old tissues with new ones for the implantation of a subsequent fertilized ovum.

Periodic endometrial thickening and endometrial ablation cause progestational tumefaction and repeated menstrual bleeding. Among mammals, this phenomenon is found only in humans and monkeys. Elaborating on the course of pregnancy, first an ovum is fertilized in the oviduct, it then descends to the uterine cavity, and finally is implanted on the thickened endometrium. During the course of pregnancy, the volume of the uterus increases considerably. Of course, the uterus returns somewhat to its original volume after delivery, but it is impossible to completely restore the original state of the uterus. Thus, the volume of the uterus is useful data for identifying a multipara.

As can be seen from the above description, the endometrium is where the fertilized fetus is implanted. If no endometrium exists in the uterus, the fertilized fetus within the uterus has no place to attach, resulting in the expulsion of the fetus. Therefore, abnormal endometrium is a major cause of sterility.

Occasionally, when the endometrium is injured due to a uterine inflammatory response, the endometrium is swept off, thereby generating endometrial synechia. In order to treat such an endometrial synechia, first an adhesion formed at the endometrium is ablated through a surgical operation, and then a certain intrauterine device, such as a loop, is inserted into the uterus to prevent reformation of the adhesion. In this case, a hormone treatment should be performed concurrently to reform endometrium. This method, of course, does not ensure a perfect treatment of sterility, but it is the best way with present medical techniques.

Among causes of sterility, where the fetus cannot be correctly implanted in the uterus due to intrauterine synechia, thereby causing sterility, first a surgeon confirms a synechia generated zone using a uterine endoscope, and then restores that zone by ablating adhesions. After that, in order to prevent regeneration of synechia, after waiting the uterus to recover for about one week, a treatment using a certain anti-synechia device should be performed concurrently with a hormone treatment using high doses of estradiol.

In the past, in order to prevent regeneration of such a synechia, the loop or catheter was installed inside the uterus after performing an adhesion ablating operation. This method, however, has several problems. That is, the shape of the loop or catheter dose not accord with the shape of the uterus, and especially the catheter cannot reach to the fundus uteri, the most important portion of the uterus, thereby reducing an effect of such a treatment by half. Furthermore, the catheter is protruded out of the vagina, resulting in inconvenience to patients. Therefore, it is required to develop a certain intrauterine device configured to conform to the shape of a woman's uterus and capable of solving the above problems.

### SUMMARY OF THE INVENTION

Therefore, the present invention has been made in view of the above problems, and it is an object of the present invention to provide an apparatus for preventing regeneration of endometrial synechia, which comprises a balloon configured to be inflated to conform to the shape of a woman's uterus by the injection of pressurized external air through an air injection tube and then an air hole, thereby preventing reformation of a uterine adhesion at the endometrium after ablation thereof and allowing a concurrent hormone treatment using certain endometrium forming hormones to be carried out.

In accordance with the present invention, the above and other objects can be accomplished by the provision of an apparatus for preventing regeneration of endometrial synechia comprising: an air injection tube, into which air is injected from the outside; a drainage lumen tube for draining intrauterine residue and blood, in which the air injection tube is received; an air hole formed at one side around an upper end of the drainage lumen tube to eject the air passed through the air injection tube; and a balloon fixed to the drainage lumen tube while covering the air hole formed at one side around the upper end of the drainage lumen tube, the balloon being configured to be inflated to conform to the shape of a woman's uterus within the uterus by the air injected through the air injection tube and air hole..

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view illustrating an apparatus for preventing regeneration of endometrial synechia in accordance with the present invention;

FIG. 2 is a plan view illustrating a state wherein a balloon according to one embodiment of the present invention is attached;

FIG. 3 is a sectional view illustrating a state wherein the balloon according to one embodiment of the present invention is attached;

FIG. 4 is a plan view illustrating a state wherein the balloon according to one embodiment of the present invention is attached and inflated inside the uterus;

FIG. 5 is a sectional view illustrating a state wherein the balloon according to one embodiment of the present invention is attached and inflated inside the uterus; and

FIG. 6 is a view illustrating a use state of the apparatus for preventing regeneration of endometrial synechia in accordance with the present invention.

### DESCRIPTION OF THE PREFERRED EMBODIMENTS

The present invention provides an apparatus for preventing regeneration of endometrial synechia, which comprises a balloon configured to be inflated, by pressurized air passed through an air injection tube and air hole, to conform to the shape of a woman's uterus, thereby maintaining the existing shape of the uterus after ablating a uterine adhesion formed due to the disruption of endometerium, and enabling a concurrent hormone treatment using oestrogen acting as an endometrium forming hormone to be carried out, thereby effectively preventing the uterine adhesion from being reformed at the endometrium.

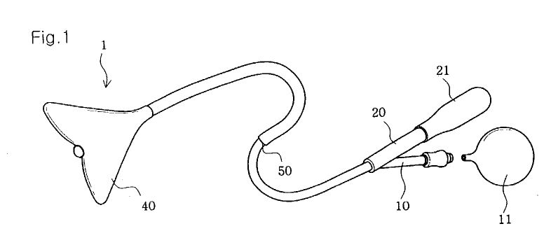

FIG. 1 is a perspective view illustrating an apparatus for preventing regeneration of endometrial synechia, applied to the present invention. As shown in FIG. 1, an apparatus for preventing regeneration of endometrial synechia, designated as a reference numeral 1, comprises an air injection tube 10, a drainage lumen tube 20, an air hole 30, and a balloon 40. According to the operation of a compressor 11 provided at the outside of the apparatus 1, external air is injected into the air injection tube 10, and then is ejected from the air hole 30 after passing through the air injection tube 10. The air injection tube 10 is received in one side of the drainage lumen tube 20, and the drainage lumen tube 20 serves to drain residue incidentally produced during a certain surgical operation. The balloon 40 is configured to be directly inserted to the fundus uteri and the isthmus of the uterine tube, thereby preventing regeneration of endometrial synechia.

Where the air injection tube 10 is connected at its distal end to the exterior compressor 11, according to the operation of the compressor 11 a constant amount of air is injected into the air injection tube 10 and then passed through the air hole 30 to pressurize the balloon 40, thereby causing the balloon 40 to be inflated to conform to the shape of a woman's uterus. The balloon 40 is formed at one side of the drainage lumen tube 20 to a thin film structure.

A diameter of the air injection tube 10 should be determined in consideration of the fact that it is received inside the drainage lumen tube 20 and it has to be inserted into a woman's uterus. Preferably, a diameter of the air injection tube 10 is about 1 to 2 millimeters.

The compressor 11, mounted at the distal end of the air injection tube 10, serves to inject external air into the air injection tube 10 in order to effect the inflation of the balloon 40, in a state wherein the intrauterine apparatus 1 is inserted into the uterus. The compressor 11 is formed so as to be separably attached to the apparatus 1 at the outside of the uterus by a surgeon.

The drainage lumen tube 20 is configured to receive the air injection tube 10, of a thin film shape, at one side therein. The drainage lumen tube 20 is used to insert the balloon 40 into the uterine cavity, after an adhesion ablating operation is performed, thereby preventing regeneration of endometrial synechia by spacing the endometrium. In addition, the drainage lumen tube 20 is used as drainage means for draining residue or blood, incidentally produced during the adhesion ablating operation, from the uterus.

A diameter of the drainage lumen tube 20, in which the air injection tube 10 is received, is preferably about 5 to 6 millimeters, so as to allow the wearer to move safely and comfortably even in a state wherein the drainage lumen tube 20 is fitted inside her uterus. The drainage lumen tube 20 may be formed at its one distal end with a residue reservoir 21 so as to temporarily store and then remove the residue or blood produced by the adhesion ablating operation.

The air hole 30 is formed at one side of the upper portion of the drainage lumen tube 20 while being connected to the air injection tube 10 received in the drainage lumen tube 20. The air hole 30 allows the air supplied through the air injection tube 10 to inflate the balloon 40 after passing there through.

The apparatus 1 for preventing regeneration of endometrial synechia further comprises a connector 50. Where the wearer has to live while fitting the apparatus 1 inside her uterus for a certain period of time after the uterine adhesion ablating operation, the connector 50 allows the wearer to comfortably use the apparatus 1 after a surgeon detaches the lower portion of the apparatus 1. Preferably, the position of the connector 50 is flexibly adjusted depending on the wearers, in consideration of a distance from the uterus to the vagina.

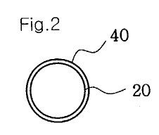

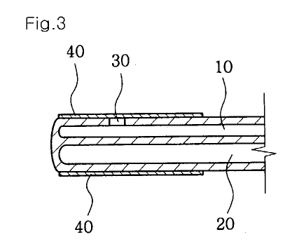

FIGS. 2 and 3 are a plan view and a front view, respectively, illustrating a state wherein the balloon 40 according to one embodiment of the present invention is attached around the outer circumference of the drainage lumen tube 20 in which the air injection tube 10 is received. As shown in FIGS. 2 and 3, the balloon 40 is attached around the outer circumference of the drainage lumen tube 20, in which the air injection tube 10 is received, by applying a certain adhesive to the drainage lumen tube 20 along a circular line. The air hole 30 is formed in a certain round region of the drainage lumen tube 20 where the adhesive is not applied, so as to allow a constant amount of the air pressurized through the air injection tube 10 and air hole 30 to inflate the balloon 40.

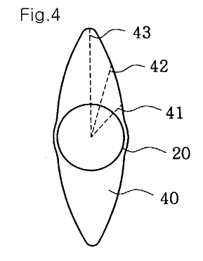

In a state wherein the balloon 40 is fixedly attached to the drainage lumen tube 20 at its both ends around one end of the drainage lumen tube 20 as stated above, the balloon 40 has a general circular shape as shown in FIG. 2. But, in an inflated state, the balloon 40 has an elliptical shape to have first through three inflated portions 41, 42 and 43 having different diameters, about the center of the drainage lumen tube 20, as shown in FIG. 4. As a result, the balloon 40 reaches the fundus uteri and the isthmus of the uterine tube and is conformed to the shape of a woman's uterus in its inflated state.

Where the balloon 40 is fixed around the one end of the drainage lumen tube 20 as shown in FIG. 3, a thickness of the upper portion of the balloon 40 attached to the drainage lumen tube 20 is thinner than that of the lower portion thereof. As a result, when the balloon 40 reaches the uterine cavity after passing through the cervix and uterine isthmus, the balloon 40 has a shape conforming to the shape of a woman's uterus, so that the balloon 40 can spread to the isthmus of the uterine tube as well as the whole interior of the uterus including the uterine isthmus and fundus uteri, thereby completely preventing regeneration of endometrial synechia.

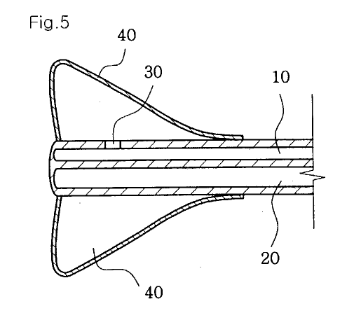

FIGS. 4 and 5 are a plan view and a front view, respectively, illustrating a state wherein the balloon 40 according to one embodiment of the present invention is inflated inside a woman's uterus while being attached to the drainage lumen tube 20. As shown in FIGS. 4 and 5, when the balloon 40 is constantly inflated by the air injected through the air hole 30, the balloon 40 is naturally curved from its upper end to its lower end. Therefore, in the inflated state thereof, the balloon 40 has a larger cross-sectional area in the upper portion thereof than in its lower portion, about the central portion formed with the air hole 30, so as to allow the inflated balloon 40 to conform to the shape of the uterus.

In use of the balloon 40 constructed as stated above, residue or blood and the like produced during a uterine adhesion ablating operation is drained to the outside through the drainage lumen tube 20.

Now, the operations and effects of the present invention constructed as stated above are explained in detail.

If endometrial synechia is generated, a uterine endoscope is inserted into the uterine cavity through the vagina and cervix to observe the interior of a women's uterus, and then an adhesion formed at the endometrium is removed using fine surgical scissors (Nd: YAG, KTP-532 or argon) or a resectoscope.

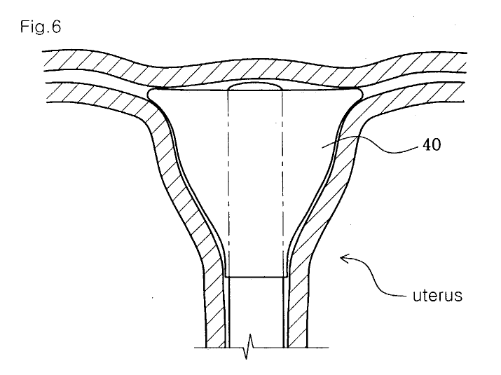

In order to prevent reformation of the adhesion after its ablating operation, the apparatus 1 for preventing regeneration of endometrial synechia according to the present invention is inserted into the uterus and then left for about 7 to 14 days as shown in FIG. 6. After the air within the air injection tube 10 is pressurized using the exterior compressor 11, the pressurized air is injected into the balloon 40 through the air hole 30 formed near the one end of the drainage lumen tube 20, thereby causing the balloon 40 to be inflated to conform to the shape of the uterus.

Since the balloon 40 inflated as stated above is highly compatible with the uterus, it can be effectively used to eliminate a cause of sterility after ablating the adhesion from the uterus of an endometrial synechia patient using the uterine endoscope. In addition, the balloon 40 can be used for stopping of bleeding after removing intrauterine submucousal leiomyoma using the endoscope.

Furthermore, the balloon 40 can be used for stopping of bleeding even in the case of cervical not-specified bleeding.

As stated above, the apparatus 1 for preventing regeneration of endometrial synechia of the present invention is characterized in that its balloon 40 is inflated to conform to the shape of the uterus, thereby uniformly spreading to the whole interior of a woman's uterus and reaching the fundus uteri. This results in minimization of inconvenience to patients.

As apparent from the above description, the present invention provides an intrauterine apparatus comprising a balloon configured to be inflated to conform to the shape of a woman's uterus, so as to be uniformly and safely spread to the whole interior of the uterus including the intrauterine isthmus and fundus uteri, thereby preventing regeneration of endometrial synechia as well as minimizing inconvenience to patients. Furthermore, the apparatus according to the present invention allows a concurrent hormone treatment to be carried out to promote formation of new endometrium.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An apparatus for preventing regeneration of endometrial synechia comprising:
an air injection tube, into which air is injected from the outside;
a drainage lumen tube for draining intrauterine residue and blood, in which the air injection tube is received;
an air hole formed at one side around an upper end of the drainage lumen tube to eject the air passed through the air injection tube; and
a balloon fixed to the drainage lumen tube while covering the air hole formed at one side around the upper end of the drainage lumen tube, the balloon being configured to be inflated to conform to a shape of a woman's uterus within the uterus by the air injected through the air injection tube and air hole.

2. The apparatus as set forth in claim 1, wherein in a state in which the balloon is attached and fixed around the upper end of the drainage lumen tube, the balloon has first through third inflated portions having different diameters from each other, thereby causing the balloon to be inflated to conform to the shape of the uterus within the uterus.

3. The apparatus as set forth in claim 2, wherein a thickness of an upper portion of the balloon attached to the drainage lumen tube is thinner than that of a lower portion thereof.

4. The apparatus as set forth in claim 2, wherein the balloon is naturally curved from its upper edge to its lower edge, thereby allowing the balloon to conform to the shape of the uterus in its inflated state.

5. The apparatus as set forth in claim 2, wherein the inflated balloon has a larger cross-sectional area in its upper portion than in its lower portion, about a central portion thereof.

6. The apparatus as set forth in claim 1, wherein a thickness of an upper portion of the balloon attached to the drainage lumen tube is thinner than that of a lower portion thereof.

7. The apparatus as set forth in claim 1, wherein the balloon is naturally curved from its upper edge to its lower edge, thereby allowing the balloon to conform to the shape of the uterus in its inflated state.

8. The apparatus as set forth in claim 1, wherein the inflated balloon has a larger cross-sectional area in its upper portion than in its lower portion, about a central portion thereof.

9. The apparatus as set forth in claim 1, further comprising:
compression means separately attached to a distal end of the air injection tube and adapted to pressurize the air, thereby causing the air to inflate the balloon after passing through the air injection tube and air hole.

10. The apparatus as set forth in claim 1, further comprising:
residue storage means detachably coupled to a distal end of the drainage lumen tube to temporarily store and then remove intrauterine residue.

11. The apparatus as set forth in claim 1, further comprising:
connecting means provided at a certain outer position of the drainage lumen tube and adapted to enable selective attachment or detachment between a lower portion of the apparatus, disposed out of the apparatus, and an upper portion of the apparatus, inserted in the uterus.
